(19) United States
(12) Patent Application Publication
(10) Pub. No.: US 2004/0158088 A1
(43) Pub. Date: Aug. 12, 2004

(54) Sequential crystallization and adsorptive refining of triglyceride oils

(75) Inventor: Ernesto Hernandez, Bryan, TX (US)

Correspondence Address: Constance Gall Rhebergen, Bracewell & Patterson, L.L.P., P.O. Box 61389, Houston, TX, 77208-1389, US

(73) Assignee: Texas A&M University

(21) Appl. No.: 10/647,809

(22) Filed: Aug. 25, 2003

Related U.S. Application Data
(60) Provisional application No. 60/405,550, filed on Aug. 23, 2002.

(51) Int. Cl. C11B 3/00; C07C 51/43

(52) U.S. Cl. 554/211

(57) ABSTRACT

A method and apparatus for refining triglyceride oils to produce haze-free, clear oils for use in cosmetics and pharmaceuticals. The method includes subjecting unrefined triglyceride oils with moderate amounts of saturated fats to a sequence of thermally regulated and adsorbent aided filtrations followed by melting, crystallization, and filtration steps to produce translucent and clear oil that can be stored for long periods of time.

### BACKGROUND OF THE INVENTION

1. Field of the Invention

This application claims the benefit of provisional patent application, U.S. Serial No. 60/405,550, filed on Aug. 23, 2002. This invention relates to the refining of triglyceride oils in the cosmetic, pharmaceutical and food industries. More particularly, this invention is directed to the removal of crystallized materials and other suspended matter from oils during refining.

2. Description of the Prior Art

Unrefined triglyceride oils contain undesirable minor components or impurities including saturated fatty acids, such as palmitic or stearic acids, and other suspended matter that, unless removed, render the oil commercially unsuitable in that they produce a haze of undesirable color, a strong flavor, or an “off” odor. Such unrefined oils are generally refined by one or several of the following steps: degumming, neutralizing or alkali refining to reduce the fatty acid content thereof, bleaching, dewaxing and deodorization.

To bleach, the neutralized oil is typically heated in the presence of bleaching clay, such as Fuller's earth, a naturally porous aluminum silicate. The oil is then subjected to a separation process after which it may be further polished and processed. The spent Fuller's earth contains from 15-50 percent by weight glyceride oil.

The main source of haziness and discoloration in triglyceride oil is the presence of crystallized triglycerides with saturated fatty acids such as palmitic or stearic acids. These triglycerides with saturated fatty acids crystallize and agglomerate producing a haze and also precipitate creating a turbid product. Another cause of haziness in the oil is the presence of other dispersed solids like protein and mucilaginous materials of microscopic size. Precipitated matter, such as proteins, can cause deterioration of the oil. When these microscopic materials agglomerate they become visible and produce unsightly haze in the final oil product. The haziness due to crystallized saturated fat is not very aesthetically pleasing. This is detrimental, particularly in cosmetics and pharmaceuticals, since it is important for the oil to be very clear and translucent to be appealing to customers.

In refining of glyceride oils, such as vegetable oils, free fatty acids are neutralized through an alkali process. Such neutralization is typically performed through the addition of a 10-15% sodium hydroxide solution to the crude oil, which also acts to hydrate gums or lecithin also present. In neutralizing the free fatty acids, “soapstock” is formed from the free fatty acids. This soapstock, or “heavy phase,” must be removed from the oil as it will otherwise inactivate bleaching clay and further deteriorates the oil during the deodorizing step. Removal is typically performed by continuous centrifugation. Phospholipids, after treatment with alkali, precipitate out with the soapstock. Sometimes the refining stage is carried out in two steps, as in the case of soybean oil processing where first the gums are separated by hydration and centrifuged and then the oil is neutralized with caustic solution and removed by centrifuging. Other alkali solutions, such as sodium bicarbonate, calcium hydroxide, potassium hydroxide, magnesium hydroxide, ammonia, and some organic bases are known in the art of alkali refining of a crude glyceride oil.

Currently, there is a commercial process that chill-proof or “winterize” vegetable oils with the purpose of producing salad oils that do not become turbid at cool temperatures. This winterization process is basically chilling of refined vegetable oils and filtrations of crystallized fat. The commercial process is used primarily with vegetable edible oils.

There is a need for an alternative to “chemical” alkali refining, such as a physical refining where oil impurities are removed by physical means. There is also a need for a separation technique of increased economic efficiency as compared to centrifugation. Centrifugation operations are usually more expensive than other physical separation techniques, produce more oil loss and sometimes require the use of a water wash. A water wash becomes necessary when saponified free fatty acids, or soap, become entrained in the oil. Under these conditions, the centrifuged oil is washed with hot water in an amount up to 15% of the oil weight. The use of extra water requires an additional centrifuging step for removal and creates a waste product that is considered a pollutant and thus is not readily disposable.

It is an object of the invention to provide a process of refining triglyceride oil wherein the undesirable components are removed from the crude triglyceride oil.

It is an object of the invention to provide a process of refining triglyceride oils wherein the undesirable components are removed from the crude triglyceride oil. Some of the objectives of this technology are to remove crystallized materials and other suspended matter to produce a clear oil that will stand long periods of storage without developing haze of discoloration.

It is a further object to provide a resulting oil that is completely clear and translucent, showing no suspended solids and no haze formation during long storage periods. It is a further object to provide an oil that can be used by itself or in combination with other ingredients in cosmetic and pharmaceutical blends, which are required to have no suspended matter or turbidity in the final product.

It is yet a further object of the invention to remove impurities from triglyceride oil through filtration.

### BRIEF SUMMARY OF THE INVENTION

The present invention advantageously includes a method and apparatus for removal of saturated fatty acids from triglyceride oils during refining through the use of an agglomerating agent that causes the contaminants to agglomerate allowing for physical separation. The method of the current invention of refining triglyceride oil to remove contaminants includes subjecting the oils to a sequence of thermally regulated and adsorbent aided-filtrations followed by melting, crystallization, and filtration steps. The method produces a translucent and clear oil that can withstand long periods of storage without developing a haze or discoloration.

Triglycerides are useful components for medicinal and cosmetic purposes, as well as food products. The contaminants most often encountered in crude oils that are agglomerated by the method of the current invention include free fatty acids. The method of the invention includes refining triglyceride oil to remove contaminants. This is accomplished by mixing an agglomerating agent with the triglyceride oil to form an oil mixture, which is then cooled. A portion of the contaminants are then removed from the oil mixture, preferably by filtration. The oil mixture is then heated such that viscosity is substantially reduced and the oil mixture is further liquefied. The oil mixture is then chilled. The oil mixture is subsequently warmed. The oil mixture is then subjected to cooling for a preselected period of time such that the oil mixture at least partially crystallizes thereby forming crystallized matter. The crystallized matter contains contaminants such as free fatty acids. A filter aid is added to the oil mixture to facilitate filtering and the crystallized matter is removed from the oil mixture through physical separation means, thereby producing a clear, refined trigylceride oil.

Physical separation is preferably performed by filtration. A filter aid may be employed to facilitate filtration. Examples of such filter aids include diatomaceous earth and Fuller's earth, among others.

The preselected time is preferably a period between five and ten hours effective to reduce the temperature of the oil mixture to between approximately 0-10 deg C. For example, cottonseed oil is cooled for approximately 8 hours so that the oil mixture is gradually cooled from room temperature to about 0-5 degrees C. Another example includes emu oil, which is cooled for 6 hours to cool from room temperature to about 5-10 deg C. This gradual cooling is performed at a predetermined rate effective to establish crystallization.

The present invention advantageously includes an apparatus for refining crude triglyceride oil to remove contaminants including a refining vessel for receiving the crude triglyceride oil and the agglomerating agent, the agglomerating agent assisting with the filtration of the contaminants within the refining vessel. Separation means is also provided to physically separate the contaminants from the triglyceride oil.

The product produced with the method of the invention is a refined triglyceride oil from crude triglyceride oil refined through contact with the agglomerating agent that acts to increase separation of phases, making filtration easier.

The method and apparatus of the present invention as well as other features, advantages, benefits and objects thereof over other methods and apparati known in the art may be better understood with reference to the detailed description which follows in conjunction with the drawings.

### BRIEF DESCRIPTION OF THE DRAWINGS

FIG. 1 is a block diagram of the prior art; and

FIG. 2 is a block diagram of a preferred embodiment of the current invention.

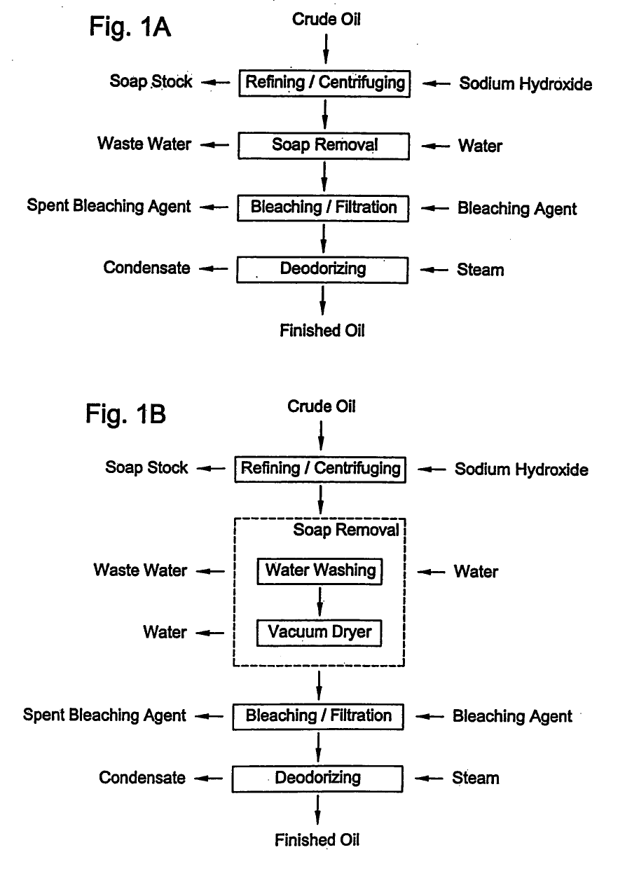

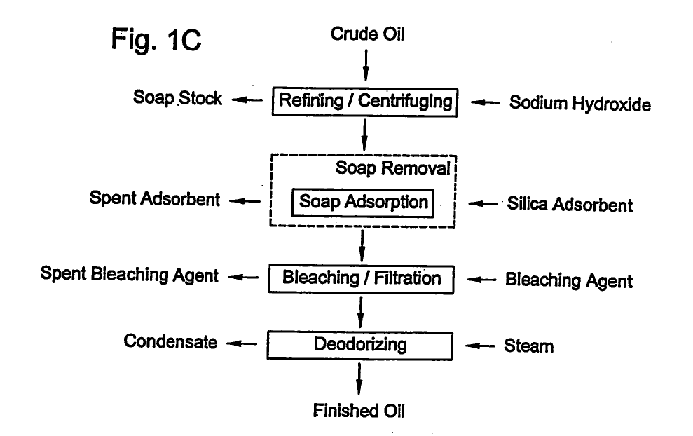

### DETAILED DESCRIPTION OF THE INVENTION

The present invention advantageously provides a method and apparatus for producing triglyceride oils that have a very clear and appealing appearance. These oils can be particularly useful for cosmetic applications. The process works particularly well with oils containing a moderate amount of saturated fatty acids, such as emu oil.

The technology in this disclosure uses sequential treatments of temperature controlled crystallization steps and a sequence of filtrations combined with filtration and adsorbents to help remove these suspended materials, i.e., crystallized triglycerides, suspended mucilaginous microscopic bodies. This invention avoids the use of alkali treatment.

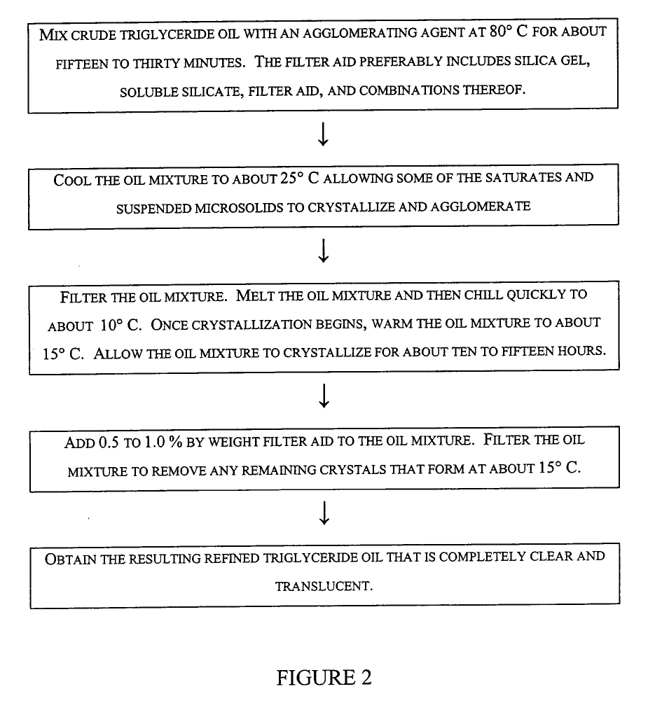

The method of the present invention refines triglycerides so that they are very clear. As shown in FIG. 2, the agglomerating agent is added to unrefined, or raw, triglyceride oil and mixed under moderate stirring conditions forming an oil mixture. The materials are preferably added to the triglyceride oil at 80° C. (176° F.). The agglomerating agent preferably includes silica gel, soluble silicate, filter aid, and combinations thereof. The oil mixture is preferably mixed for about fifteen to thirty minutes and then cooled to a temperature in the range of about 0 degrees C. to 35 degrees C., more preferably about 25° C. (77° F.). Cooling allows some of the saturates and suspended micro-solids to crystallize and agglomerate. In a preferred embodiment, this cooling is performed in a substantially absence of mechanical mixing.

In a preferred embodiment, the mixing of the agglomerating agent is performed while the oil mixture is in a temperature range of 60 to 100 deg C. and for a period of time between 15 and 30 minutes.

Silicate solutions react with the free fatty acids in crude oils acting to neutralize the solution. The contaminants readily agglomerate, which allows for better filtration. This also serves as a “bleaching” and adsorbent pretreatment of the oils by removing color impurities, thus providing a simplified method for refining of crude triglyceride oil. Effective liquid refining agents include soluble silicate solutions, with sodium metasilicate being a preferred silicate. The agglomerated fatty acids are a mesh of interlocking neutralized free fatty acids and silica gel produced by the introduction of the soluble silicate solution. In effect, silica gel is produced in situ binding the contaminants. Other contaminants present in the triglyceride oil will be contained in this discrete phase. The invention creates a clear boundary definition for the discrete phase when silica gel is formed with the interaction of the crude oil with contaminants with the agglomerating agent.

Preferred silicate solutions include silica gel, a soluble silicate, amorphous silica, a filter aid, and combinations of the same. Notably, powdered silicates are difficult to mix into the oil mixture.

Once the mixture is cool, it is then filtered. The filtered mixture is then completely melted, which liquefies all oil within the mixture, and then chilled quickly to about 5-15° C. (59° F.). Once crystallization has started, the oil mixture is then warmed up to 10-20° C. (68° F.). The oil mixture is then allowed to crystallize for a period of four to fifteen hours. Following the crystallization phase, 0.5 to 1.0 percent by weight of filter aid is added to the oil mixture. The oil mixture is then filtered, which removes the remaining crystals that form at 5-15° C. (59° F.).

The resulting oil is completely clear and translucent, shows no suspended solids and no haze formation. The oil remains clear and haze-free even after long periods of storage. Samples of oils, such as Emu oil, refined by the methods described herein have remained clear during storage of up to 8 months in a shelf at cool temperatures (above 18° C. (64° F.)), without showing turbidity or precipitation.

The resulting oil also meets specifications for refined oil such that the refined triglyceride oil contains no more than 0.05% free fatty acids by weight. Advantageously, the method and apparatus of the invention allow for the creation of refined oil that contains no more that 0.03% free fatty acids by weight.

The resulting product oil can be used by itself or in combination with other ingredients, such as in cosmetic and pharmaceutical blends that are required to have no suspended matter or turbidity in the final product. This quality exceeds normal food product quality.

The method of the present invention, effective for use with triglyceride oils derived from animal sources, works particularly well with emu oil. Emu oil, which contains triglycerides, is currently sold as a specialty oil for cosmetic and pharmaceutical applications. The presence of suspended matter and turbidity is one factor that makes this oil less appealing and more difficult to handle for some applications, especially in cosmetics. Vegetable oils, such as cottonseed and palm oil, are also useful in the current invention.

Emu oil possesses many desirable characteristics that are especially useful in the cosmetic and pharmaceutical industry. As more research is performed on emu oil, more benefits are becoming known about the oil. Emu oil is a healthy, complex, topical or ingestible oil that is trans-dermal. Emu oil contains skin penetrating, moisturizing, anti-arthritic and anti-inflammatory properties. Emu oil is also used as carrier of pharmaceuticals such ethyl salicylate, and nutraceutical oils, such as oil of eucalytptus.

Emu oil can alleviate many common problems. In addition to reducing swelling and stiffness in joints, it reduces bruising and muscle pain. Professional sports trainers and players recognize the benefits of emu oil. Emu oil is a natural emollient. Emu oil helps combat the effects of the aging process. Research has shown emu oil can thicken the skin, which reduces the appearance of aging. Medical specialists are discovering the benefits of emu oil and are adapting it into their treatment techniques.

In addition to the usefulness of the oil in cosmetics and pharmaceuticals, there are other benefits associated with use of this oil. Emu oil is a natural, environmentally friendly product, which makes issues such as disposal, handling, toxicity, and the like much easier to manage.

For certain products, emu oil is required to be clear and translucent to give it a more appealing appearance and provide better handling. The process of the present invention provides a clear triglyceride oil and works particularly well with emu oil. Furthermore, the lack of alkali processing serves to preserve the specific components present in unrefined oil that possess particularly beneficial qualities, such as vitamin E in vegetable oils.

The apparatus of the current invention includes means for introducing a silicate solution into a crude oil, with a preferred crude oil being triglyceride oil. Preferably, the silicate solution is a non-dilute solution. Means for heating the silicate-treated crude oil is provided such that the silicate-treated crude oil is heated to a temperature at which agglomeration occurs such that the silicate-treated crude oil provides the discrete phase, which acts as a pseudo-solid phase, and a liquid oil phase. A filtration device receives the silicate-treated crude oil for separation of the solid phase from the liquid phase, such liquid phase including refined glyceride oil. The apparatus of the invention for refining crude triglyceride oil to remove contaminants includes a refining vessel for receiving the crude triglyceride oil and an agglomerating agent. The crude triglyceride oil and agglomerating agent combine to form an oil mixture. The agglomerating agent causing the contaminants in the oil mixture to agglomerate within the refining vessel into fatty crystals. Also included is a mixing means operable to mix the agglomerating agent with the crude triglyceride oil. A cooling means is advantageously provided to cool the oil mixture. The cooling means is operable to cool the oil mixture to a predetermined temperature range in a predetermined amount of time. The apparatus also includes a warming means operable to warm the oil mixture. Separation means is also provided to physically separate agglomerated contaminants from the oil mixture to produce the refined oil.

Commercial filtration means can include rotary filter, filter press or leaf filter, filter cloth, metal gauze or any other variety of filtration method. The filter cake produced by filtration means is a waste product of this system. Through the use of the silicate solution, the filter cake contains relatively little residual oil, thus minimizing disposal problems. As use of the silicate solution avoids the need of a water wash, no contaminated water stream exists for disposal.

Advantages achieved by the method of the invention include a reduction in processing costs due to the elimination of the need for centrifuge equipment. A water wash step is eliminated as most fatty acids are eliminated in the filtration step.

From the foregoing it will be seen that this invention is one well adapted to attain all of the ends and objects hereinabove set forth, together with other advantages which are obvious and which are inherent to the apparatus and structure.

It will be understood that certain features and subcombinations are of utility and may be employed without reference to other features and subcombinations. This is contemplated by and is within the scope of the claims.

Because many possible embodiments may be made of the invention without departing from the scope thereof, it is to be understood that all matter herein set forth or shown in the accompanying drawings is to be interpreted as illustrative and not in a limiting sense.

For example, the surprising result of solidification upon increased temperature and concentration of the neutralizing solution allows for variation of processing conditions while still maintaining these features. Such variations of processing conditions are encompassed within this invention. Various silicates perform the same function at different conditions and are thus fairly within the scope of the invention. Silicate, while described as a solution, can be provided in any form, including solid, such that a solution is formed with the glyceride oil. Further additives can be used to harden or compress the agglomerated solids or to permit maintenance of such flocs over extended periods, relative to processing needs.

We claim:

1. A method of refining triglyceride oil to remove contaminants, comprising the steps of:
mixing an agglomerating agent with the triglyceride oil to form an oil mixture;
cooling the oil mixture;
removing at least a portion of the contaminants from the oil mixture;
heating the oil mixture;
chilling the oil mixture;
warming the oil mixture;
cooling the oil mixture for a preselected period of time such that the oil mixture at least partially crystallizes thereby forming crystallized matter, the crystallized matter containing contaminants;
adding a filter aid to the oil mixture to facilitate filtering; and
filtering the crystallized matter from the oil mixture, thereby producing a refined trigylceride oil.

2. The method of claim 1 wherein the preselected time is a period between five and ten hours effective to reduce the temperature of the oil mixture to between approximately 0-10 degrees C.

3. The method of claim 1 wherein the step of removing at least a portion of the contaminants is performed through filtration.

4. The method of claim 1 wherein the refined triglyceride oil contains no more than 0.05% free fatty acids by weight.

5. The method of claim 1 wherein the refined triglyceride oil contains no more that 0.03% free fatty acids by weight.

6. The method of claim 1 wherein the step of heating the oil mixture is effective to liquefy at least a majority of the oil mixture.

7. The method of claim 1 wherein the step of heating the oil mixture is effective to substantially reduce the viscosity of the oil mixture.

8. The method of claim 1 wherein the cooling of the oil mixture is at a predetermined rate effective to establish crystallization.

9. The method of claim 1 wherein the contaminants include free fatty acids.

10. The method of claim 1 wherein the agglomerating agent comprises a soluble silicate solution.

11. The method of claim 1 wherein the agglomerating agent is selected from the group consisting of silica gel, a soluble silicate, amorphous silica, a filter aid, and combinations thereof.

12. The method of claim 1, wherein the filter aid is selected from the group consisting of diatomaceous earth and Fuller's earth.

13. The method of claim 1 wherein the triglyceride oil comprises a vegetable oil.

14. The method of claim 1 wherein the triglyceride oil comprises oil derived from an animal source.

15. The method of claim 1 wherein the triglyceride oil comprises emu oil.

16. The method of claim 1 wherein the mixing of the agglomerating agent is performed while the oil mixture is in a temperature range of 60 to 100 deg C. and for a period of time between 15 and 30 minutes.

17. The method of claim 1 wherein the step of cooling the oil mixture results in the oil mixture having a temperature in the range of 0 deg C. to 35 deg C.

18. The method of claim 1 wherein the step of cooling the oil mixture is performed in a substantial absence of mechanical mixing.

19. An apparatus for refining crude triglyceride oil to remove contaminants, comprising:
a refining vessel for receiving the crude triglyceride oil and an agglomerating agent to create an oil mixture, the agglomerating agent causing the contaminants in the oil mixture to agglomerate within the refining vessel;
a mixing means operable to mix the agglomerating agent with the crude triglyceride oil;
a cooling means to cool the oil mixture, the cooling means being operable to cool the oil mixture to a predetermined temperature range in a predetermined amount of time;
a warming means operable to warm the oil mixture; and
a separation means to physically separate agglomerated contaminants from the oil mixture.

20. The apparatus of claim 19 wherein the cooling means is operable to establish crystallization in the oil mixture, the crystallization operable to create crystals containing at least a portion of the contaminants.

21. The apparatus of claim 19 wherein the contaminants include free fatty acids.

22. The apparatus of claim 19 wherein the agglomerating agent comprises a silica compound.

23. The apparatus of claim 19 wherein the agglomerating agent is selected from the group consisting of silica gel, a soluble silicate, a filter aid, and combinations thereof.

24. The apparatus of claim 19, wherein the filter aid is selected from the group consisting of diatomaceous earth and Fuller's earth.

25. The apparatus of claim 19 wherein the triglyceride oil comprises oil derived from an animal source.

26. The apparatus of claim 19 wherein the triglyceride oil comprises emu oil.

27. The apparatus of claim 19 wherein the mixing means is effective to substantially mix the agglomerating agent into the triglyceride oil while the oil mixture is in a temperature range of 60 to 100 deg C. during a period of time between 15 and 30 minutes.

28. The apparatus of claim 19 wherein the cooling means is operable to cool the oil mixture to a temperature in the range of 0 deg C. to 35 deg C.

29. The apparatus of claim 19 wherein the mixing means is operable to suspend mixing during operation of the cooling means.

30. A refined triglyceride oil comprising crude triglyceride oil refined through contact with an agglomerating agent, the agglomerating agent being mixed with the crude triglyceride oil to form an oil mixture, the oil mixture being cooled for a preselected period of time such that the oil mixture crystallizes thereby forming crystallized matter containing contaminants, a filter aid being added to the oil mixture to facilitate physical separation and physical separation means to remove crystallized matter to produce the refined triglyceride oil.
